(19) United States
(12) Patent Application Publication
(10) Pub. No.: US 2015/0216957 A1
(43) Pub. Date: Aug. 6, 2015

(54) PLACENTAL VACCINATION THERAPY FOR CANCER

(71) Applicant: Boris Markosian, Burbank, CA (US)

(72) Inventor: Boris Markosian, Burbank, CA (US)

(21) Appl. No.: 14/422,644

(22) PCT Filed: Aug. 19, 2013
(86) PCT No.: PCT/US2013/055632

§ 371 Date: Feb. 19, 2015

Related U.S. Application Data
(60) Provisional application No. 61/691,187, filed on Aug. 20, 2012.

(51) Int. Cl. A61K 39/00

(52) U.S. Cl. CPC A61K 39/0011; A61K 2039/5154; A61K 2039/51; A61K 2039/55538; A61K 2039/55561; A61K 2039/55522; A61K 2039/55505; A61K 2039/55566

(57) ABSTRACT

The present invention discloses methods of stimulating an immune response to cancer tissue and biological effectors used by tumors to defeat the host, through the immunization of a mammal using placental tissue, cells, molecules, and combinations thereof. In one embodiment, the invention provides the utilization of syngeneic, allogeneic, xenogeneic, and combinations thereof as immunizing sources.

### PRIORITY CLAIM

This application claims priority to U.S. Provisional Application No. 61/691,187, filed Aug. 20, 2012, the contents of which are incorporated by reference herein in their entirety.

The present invention discloses methods of stimulating an immune response to cancer tissue and biological effectors used by tumors to defeat the host, through the immunization of a mammal using placental tissue, cells, molecules, and combinations thereof. In one embodiment, the invention provides the utilization of syngeneic, allogeneic, xenogeneic, and combinations thereof as immunizing sources.

### DESCRIPTION OF THE INVENTION

### Field of the Invention

The invention disclosed pertains to the field of immune modulation. More specifically, the invention pertains to a bi-functional preparation that concurrently augments immune function while containing therapeutically useful antigens.

### BACKGROUND OF THE INVENTION

Surgery, radiation therapy, and chemotherapy have been the standard accepted approaches for treatment of cancers including leukemia, solid tumors, and metastases. Unfortunately, these approaches are associated with extremely high toxicity and adverse effects. Immunotherapy which uses the body's immune system, either directly or indirectly, to shrink or eradicate cancer has been studied for many years as an adjunct to conventional cancer therapy. It is believed that the human immune system may be an untapped resource for cancer therapy and that effective treatment can be developed once the components of the immune system are properly harnessed. As key immunoregulatory molecules and signals of immunity are identified and prepared as therapeutic reagents, the clinical effectiveness of such reagents can be tested using established cancer models. Immunotherapeutic strategies include administration of vaccines, activated cells, antibodies, cytokines, chemokines, as well as small molecular inhibitors, anti-sense oligonucleotides, and gene therapy. The invention provided herein provides a novel method for the treatment of cancer without the toxicities associated with current approaches to cancer therapy.

### BRIEF SUMMARY OF THE INVENTION

The invention disclosed teaches a novel method of inducing anti-tumor and immune stimulating effects in a host through immunization with xenogeneic placenta, placental extracts, cells, cell lines or purified protein combinations. A few advantages of a xeno-placental vaccination approach over allo-placental vaccination reside in the unexpected potent properties of the xenoantigenic composition to stimulate immune responses against: (1) tumor associated antigens; (2) functional tumor associated antigens; and (3) tumor secreted immune suppressive components. The utility and practicality of the invention disclosed lends itself to treatment of other immune suppression-associated states in which the host requires immune stimulation.

In one aspect of the invention, allogeneic placenta is utilized but immunogenicity is augmented by pre-treatment of said placental tissue, or cells thereof with agents capable of stimulating immunogenicity. Agents capable of stimulating immunogenicity include, but are not limited to, activators of MHC expression such as cytokines, histone deacetylase inhibitors and DNA methyltransferase inhibitors. Additional means of immune modulation include transfection of cells with xenogeneic or allogeneic components, as well as transfected with immune stimulatory cytokines.

Non-limiting examples of immune stimulatory cytokines include B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), I-309, Intercellular Adhesion Molecule 1 (“ICAM-1”), Interferon gamma (“IFN-gamma”), Interlukin-1 alpha (“IL-1 alpha”), Interlukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C-C motif) Lignad 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1alpha (“SDF-1 alpha”), Chemokine (C-C motif) ligand 17 (“TARC”), Thymus-expressed chemokine (“TECK”), Thymic stromal lymphopoietin (“TSLP 4-1BB”), CD166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-beta1, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member 10C (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein 1B (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, Follistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD163, Clusterin, CRTAM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κ B (“RANK”).

In another aspect of the invention, a cancer vaccine comprising pluripotent stem cells differentiated towards the trophoblastic lineage is used, wherein said pluripotent stem cells are defined as cells capable of differentiating into mesoderm, ectoderm and endodermal tissues and furthermore wherein said pluripotent stem cells are selected from the following group of cells: (a) embryonic stem cells; (b) parthenogenic derived stem cells; (c) inducible pluripotent stem cells; (d) somatic cell nuclear transfer derived stem cells.

In another aspect of the invention, the cancer vaccine comprises a trophoblastic lineage cell optimized for expression of HLA-G, CD146, progesterone, placental growth factor, and placental lactogen. The invention further provides the use of trophoblast lineage committed cells generated from pluripotent stem cells, wherein the cells are differentiated by treatment of the pluripotent stem cells with BMP-4 in the absence of FGF-2. Alternatively, differentiation may be performed by exposing the pluripotent cells to inhibitors of activin and FGF2 signaling while providing BMP-4.

In another aspect of the invention, differentiation towards trophoblast lineage is performed by exposing said pluripotent cells to inhibitors of activin and FGF2 signaling while providing BMP-4 in the presence of a feeder layer of cells. In this embodiment of the invention, the feeder layer is substantially comprised of cells selected from a group comprising: (a) mammalian embryonic fibroblast cells; (b) mammalian keloid tissue derived cells; (c) mammalian endothelial cells; and (d) mammalian mesenchymal stem cells. In another aspect of the invention, trophoblast differentiated cells are exposed to an agent capable of increasing immunogenicity, wherein the agent capable of upregulating expression of MHC and/or one or more co-stimulatory molecules on the trophoblast differentiated cells.

In yet another aspect of the invention, a cancer vaccine is provided in which trophoblasts have been treated with an agent capable of increasing immunogenicity induces or substantially upregulates expression of a molecule selected from a group of molecules comprising: (a) CD80; (b) CD86; (c) CD40; (d) ICAM-1; (e) LFA-3; and (f) IL-12.

In another aspect of the invention, trophoblast, or trophoblast-like cells are transfected with genes encoding molecules to increase immunogenicity, said molecules are selected from: (a) ABCF1, (b) TNF-alpha; (c) TNF-beta; (d) BCL6, (e) complement C3; (f) complement C4A; (g) complement C5; (h) CEBPB; (i) CRP, (j) ICEBERG, (k) IL1R1, (1) IL1RN; (m) IL8RB; (n) LTB4R; (o) TOLLIP; (p) IFNA2; (q) IL12; (r) IL13RA1; (s) CD40L; (t) IFNA2; (u) IL17C; (v) IL18; (w) IL-21; (x) IL-22; (y) G-CSF; (z) GM-CSF; (aa) interferon gamma; (ab) the whole or components of the HLA protein; (ac) the whole or components of allogeneic HLA protein; and (ad) the whole or components of xenogeneic HLA proteins.

In one aspect of the invention, the cells utilized for immunization, whether placental derived trophoblasts, pluripotent derived trophoblasts, or trophoblasts are created by cellular reprogramming, are allogeneic, xenogeneic or syngeneic to the recipient. The recipient is a mammal including homo sapien, canine domesticus, feline, equine or other mammals. In one aspect of the invention, a cancer vaccine is provided where the vaccine comprises trophoblast or endothelial cells which are transfected with alpha 1,3 galactosyl transferase so as to induce expression of sufficient amount of the terminal carbohydrate epitope Gal alpha(1,3)Gal. In one specific aspect of the invention, the cancer vaccine provided contains alpha 1,3galactosyl transferase gene that has been transfected at a level sufficient such that transfected cells are capable of inducing activation of complement cascade in the presence of human blood. In one aspect of the invention, a trophoblast-like cell is generated from a pluripotent stem cell, wherein the trophoblast-like cell is optimized for expression of antigens representing tumor biological activities. In one aspect of the invention, a trophoblast cell used for tumor vaccination is differentiated from said pluripotent stem cell so as to express biological activities selected from a group of biological activities including: (a) invasiveness; (b) immune evasion; and/or (c) angiogenesis.

### BRIEF DESCRIPTION OF THE FIGURES

FIGS. 1-3 represent prophylaxis of B-16 tumor growth in C57/BL6 mice immunized with porcine placental vaccine (xenogeneic), but not porcine liver extract or control, according to one embodiment of the invention.

FIGS. 4-6 represents immunization with xenogeneic placental extracts using syngeneic DC as adjuvants according to one embodiment of the invention.

FIG. 7 illustrates the synergy between B16 cell lysate vaccination and xenogeneic placental vaccination according to one embodiment of the invention.

FIG. 8 illustrates the therapeutic effects of xenogeneic placental extract on treatment of colon cancer mouse model according to one embodiment of the invention.

### DETAILED DESCRIPTION OF THE INVENTION

Unless otherwise specified, the definitions and scientific terminology used in this document possess the meanings that those of skill in the art would interpret as common knowledge. The methodology thought or referenced in this document is generally well understood and commonly employed by those skilled in the art. Furthermore, unless defined differently, all technical and scientific terms used herein have the same meanings as commonly understood by one of skill in the art to which the disclosed invention belongs. In particular, the following terms and phrases have the following meaning.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells.

The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention.

Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Non-limiting examples of cancers are cancer of the brain, melanoma, bladder, breast, cervix, colon, head and neck, kidney, lung, non-small cell lung, mesothelioma, ovary, prostate, sarcoma, stomach, uterus and medulloblastoma.

The term “leukemia” is meant broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

The term “carcinoma” refers to a malignant new growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti,

The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

The term “polypeptide” is used interchangeably with “peptide”, “altered peptide ligand”, and “flourocarbonated peptides.”

The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions described herein.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an alpha, beta T cell expressing TCR alpha and beta chains, and a gamma delta T cell expressing TCR gamma and delta chains. The T cell includes a precursor cell of a T cell in which differentiation into a T cell is directed.

Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids.

Further, a variety of cell populations containing T cells and derived from hematopoietic cells can be used in the present invention. In various embodiments, these cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used. The term “antibody” is meant to include both intact molecules as well as fragments thereof that include the antigen-binding site. Whole antibody structure is often given as H₂L₂ and refers to the fact that antibodies commonly comprise 2 light (L) amino acid chains and 2 heavy (H) amino acid chains. Both chains have regions capable of interacting with a structurally complementary antigenic target.

The regions interacting with the target are referred to as “variable” or “V” regions and are characterized by differences in amino acid sequence from antibodies of different antigenic specificity. The variable regions of either H or L chains contain the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure.

The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains.

The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered.

The terms “individual”, “host”, “subject”, and “patient” are used interchangeably herein, and refer to a mammal, including, but not limited to, primates, for example, human beings, as well as rodents, such as mice and rats, and other laboratory animals.

As used herein, the term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (co-stimulatory signal) upon exposure of a T cell and/or NK cell to an antigen.

The term “enhanced function of a T cell”, “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization.

“Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and most preferably 90% or more.

A trophoblast is a cell which is a precursor of the cells which participate in the formation of the human placenta. When an embryo begins differentiation, at the stage of a blastocyst, the cells in the inner cell mass are committed to form the cells which will become the embryo, while the outer cells of the blastocyst become committed to participate in the development of the placenta. Human trophoblast cell lines have been created from transformed placental cells, which may be useful for the practice of the invention. For the practice of the current invention, “trophoblasts” include relatively undifferentiated villous cytotrophoblast, intermediate cytotrophoblast, terminally differentiated villous syncytiotrophoblast and extravillous cytotrophoblast that invade into maternal decidua. These differentiated trophoblasts arise from a putative trophoblast stem cell population; it has been proposed that at the villous basement membrane contains a cell population.

In one embodiment of the invention, chorionic villous tissues from patients who voluntarily chose to terminate pregnancy during the first trimester (between 6^{th} and 9^{th} week of gestation) are obtained. Villous cytotrophoblast cells are isolated by finely mincing and dissociating tissue in Hanks' balanced salt solution (HBSS) containing HEPES (25 mmol), DNase1 and collagenase (15 U/ml) (Sigma, St. Louis, USA) for 30 min at 37° C. with agitation. The dispersed cells are separated by filtration through a wire sieve (40 μm diameter pores) and stored in the presence of 10% FCS. After percoll gradient centrifugation, cells are obtained on the order of 1×10⁶ mono-dispersed cells. Cells may be cultured in a variety of media, in one example cytotrophoblast cells are cultured in RPMI media with 10% fetal calf serum with 1% penicillin and streptomycin. Other additives to culture may be used, for example 2 mM L-glutamine, 100 uM 2-mercaptoethanol, and murine embryonic fibroblast conditioned media.

As used herein, the use of the word “a” or “an” when used in conjunction with the term “comprising” in the claims and/or the specification may mean “one,” but it is also consistent with the meaning of “one or more,” “at least one,” and “one or more than one.”

The terms “having,” “including,” “containing,” and “comprising” are interchangeable and one of skill in the art is cognizant that these terms are open ended terms.

“Embryonic germ cells” or “EG cells” are cells derived from the primordial germ cells of an embryo or fetus that are destined to give rise to sperm or eggs. EG cells are among the embryonic stem cells that can be cultured in accordance with the invention.

“Embryonic stem cells” or “ES cells” are cells obtained from an animal (e.g., a primate, such as a human) embryo, preferably from an embryo that is less than about eight weeks old. Preferred embryonic stages for isolating primordial embryonic stem cells include the morula or blastocyst stage of a pre-implantation stage embryo. Well-known criteria for characterizing a cell as a stem cell are intended herein. See, e.g., Hoffman and Carpenter, Nature Biotech. 23:699-708, 2005, which is incorporated by reference in its entirety.

“Extracellular matrix” (ECM) or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. The ECM thus encompasses essentially all secreted molecules that are immobilized outside of the cell. In vivo, the ECM provides order in the extracellular space and serves functions associated with establishing, separating, and maintaining differentiated tissues and organs. The ECM is a complex structure that is found, for example, in connective tissues and basement membranes, also referred to as the basal lamina. Connective tissue typically contains isolated cells surrounded by ECM that is naturally secreted by the cells. Components of the ECM have been shown to interact with and/or bind growth and differentiation factors, cytokines, matrix metalloproteases (MMPs), tissue inhibitors of metalloproteases (TIMPs), and other soluble factors that regulate cell proliferation, migration, and differentiation. Descriptions of the ECM and its components may be found in, among other places, Guidebook to the Extracellular Matrix, Anchor, and Adhesion Proteins, 2d ed., Kreis and Vale, eds., Oxford University Press, 1999 (“Kreis et al.”); Geiger et al., Nature Reviews Molecular Cell Biology 2:793-803, 2001; Iozzo, Annual Review of Biochemistry, 1998, Annual Reviews, Palo Alto, Calif.; Boudreau and Jones, Biochem. J. 339:481-88, 1999; Extracellular Matrix Protocols, Streuli and Grant, eds., Humana Press 2000; Metzler, Biochemistry the Chemical Reactions of Living Cells, 2d ed., vol. 1, 2001, Academic Press, San Diego, each of which are incorporated by reference in their entirety.

“Pluripotent” refers to cells that are capable of differentiating into one of a plurality of different cell types, although not necessarily all cell types. A non-limiting exemplary class of pluripotent cells is embryonic stem cells, which are capable of differentiating into any cell type in the human body. Thus, it will be recognized that while pluripotent cells can differentiate into multipotent cells and other more differentiated cell types, the process of reverse differentiation (i.e., de-differentiation) is likely more complicated and requires “re-programming” the cell to become more primitive, meaning that, after re-programming, it has the capacity to differentiate into more or different cell types than was possible prior to re-programming. Stem cells, including primate primordial stem cells, cultured in accordance with the invention can be obtained from any suitable source using any appropriate technique. For example, procedures for isolating and growing human primordial stem cells are described in U.S. Pat. No. 6,090,622. Procedures for obtaining Rhesus monkey and other non-human primate primordial stem cells are described in WO 96/22362. In addition, methods for isolating Rhesus monkey primordial stem cells are described by Thomson, et al., Proc. Natl. Acad. Sci. USA 92:7844-7848, 1995.

“Stem cell” includes any stem or precursor cell, whether from a human or non-human source, and cells derived from stem cells that retain characteristics of precursor cells. Human embryonic stem cells (hESCs) can be isolated, for example, from human blastocysts obtained from human in vivo preimplantation embryos, in vitro fertilized embryos, or one-cell human embryos expanded to the blastocyst stage. See, e.g., Bongso, et al. (1989), Hum. Reprod. 4:706.

Human embryos can be cultured to the blastocyst stage in G1.2 and G2.2 medium. See, e.g., Gardner, et al., Fertil. Steril. 69:84 (1998). The zona pellucida is removed from blastocysts by brief exposure to pronase (Sigma). The inner cell masses can be isolated by immunosurgery or by mechanical separation, and are plated on mouse embryonic feeder layers, or in the defined culture system as described herein. After nine to fifteen days, inner cell mass-derived outgrowths are dissociated into clumps either by exposure to calcium and magnesium-free phosphate-buffered saline (PBS) with 1 mM EDTA, by exposure to dispase, collagenase, or trypsin, or by mechanical dissociation with a micropipette. The dissociated cells are then replated as before in fresh medium and observed for colony formation. Colonies demonstrating undifferentiated morphology are individually selected by micropipette, mechanically dissociated into clumps, and re-plated.

Embryonic stem cell-like morphology is characterized as compact colonies with apparently high nucleus to cytoplasm ratio and prominent nucleoli. Resulting embryonic stem cells are then routinely split every 1-2 weeks by brief trypsinization, exposure to Dulbecco's PBS (without calcium or magnesium and with 2 mM EDTA), exposure to type IV collagenase (about 200 U/mL), or by selection of individual colonies by mechanical dissociation, for example, using a micropipette.

Stem cells are undifferentiated cells defined by their ability at the single cell level to both self-renew and differentiate to produce progeny cells, including self-renewing precursors, non-renewing precursors and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent—able to give rise to all embryonic and extraembryonic cell types; (2) pluripotent—able to give rise to all embryonic cell types; (3) multipotent—able to give rise to a subset of cell lineages, but all within a particular tissue, organ, or physiological system (for example, hematopoietic stem cells (HSC) can produce progeny that include HSC (self-renewal), blood cell-restricted oligopotent precursors, and all cell types and elements (e.g., platelets) that are normal components of the blood); (4) oligopotent—able to give rise to a more restricted subset of cell lineages than multipotent stem cells; and (5) unipotent—able to give rise to a single cell lineage (e.g., spermatogenic stem cells).

Cancer refers to various types of malignant neoplasms and tumors, including primary tumors, and tumor metastasis. Non-limiting examples of cancers which can be detected by the sensor array and system of the present invention are brain, ovarian, colon, prostate, kidney, bladder, breast, lung, oral, and skin cancers. Specific examples of cancers are: carcinomas, sarcomas, myelomas, leukemias, lymphomas and mixed type tumors. Particular categories of tumors include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma.

“Substantially undifferentiated” means that population of stem cells (e.g., primate primordial stem cells) contains at least about 50%, preferably at least about 60%, 70%, or 80%, and even more preferably, at least about 90%, undifferentiated, stem cells. Fluorescence-activated cell sorting using labeled antibodies or reporter genes/proteins (e.g., enhanced green fluorescence protein or EGFP) to one or more markers indicative of a desired undifferentiated state (e.g., a primordial state) can be used to determine how many cells of a given stem cell population are undifferentiated. For purposes of making this assessment, one or more of cell surface markers correlated with an undifferentiated state (e.g., Oct-4, SSEA-4, Tra-1-60, and Tra-1-81) can be detected.

Telomerase reverse transcriptase (TERT) activity and alkaline phosphatase can also be assayed. In the context of primate primordial stem cells, positive and/or negative selection can be used to detect, for example, by immuno-staining or employing a reporter gene (e.g., EGFP), the expression (or lack thereof) of certain markers (e.g., Oct-4, SSEA4, Tra-1-60, Tra-1-81, SSEA-1, SSEA-3, nestin, telomerase, Myc, p300, and Tip60 histone acetyltransferases, and alkaline phosphatase activity) or the presence of certain post-translational modifications (e.g., acetylated histones), thereby facilitating assessment of the state of self-renewal or differentiation of the cells.

“Totipotent” refers to cells that are capable of differentiating into any cell type, including pluripotent, multipotent, and fully differentiated cells (i.e., cells no longer capable of differentiation into various cell types), such as, without limitation, embryonic stem cells, neural stem cells, bone marrow stem cells, hematopoietic stem cells, cardiomyocytes, neuron, astrocytes, muscle cells, and connective tissue cells. General methods in molecular genetics and genetic engineering are described in the current editions of “Molecular Cloning: A Laboratory Manual” (Sambrook, et al., Cold Spring Harbor); Gene Transfer Vectors for Mammalian Cells (Miller & Calos eds.); and “Current Protocols in Molecular Biology” (Ausubel, et al. eds., Wiley & Sons). Cell biology, protein chemistry, and antibody techniques can be found in“Current Protocols in Protein Science” (Colligan, et al. eds., Wiley & Sons); “Current Protocols in Cell Biology” (Bonifacino, et al., Wiley & Sons) and “Current Protocols in Immunology” (Colligan et al. eds., Wiley & Sons.). Reagents, cloning vectors, and kits for genetic manipulation referred to in this disclosure are available from commercial vendors such as BioRad, Stratagene, Invitrogen, ClonTech, and Sigma-Aldrich Co.

“About” as used herein when referring to a measurable value such as an amount, a temporal duration, and the like, is meant to encompass variations of ±20%, ±10%, ±5%, ±1%, and ±0.1% from the specified value, as such variations are appropriate to perform the disclosed methods.

Specific embodiments of the invention will be described below. An embodiment of the invention teaches the utilization of xenogenic placenta as a source of immune suppressive and tumor antigens. Through immunization of the recipient using xenogeneic placenta or products derived from, an immune response can be stimulated against immune suppressive factors and tumor antigens associated with tumors. This immune response is capable of neutralizing endogenous immune suppressants that possess homology with the immune suppressive compounds found in the placenta. It was discovered that the xenogeneic component of the preparation is important for stimulating a more robust immune response compared to immunization with allogeneic placental tissue. Specifically, a host suffering from cancer may be immunized with placental extract purified according to methods known in the art. Specific application of the invention may be, but is not limited to, utilization of xenogeneic placenta from a porcine source for immunization into a human cancer patient. Placental tissue obtained from delivering sows is obtained and washed in sterile phosphate buffered saline containing 5% penicillin streptomycin mixture. Subsequently, the placental tissue is immediately homogenized in sterile saline placed in 50-ml tubes (˜1 g of tissue/tube) using an electronic homogenizer (Ultra Turrax T25-S1; Janke&Kunkel KG, Staufen, Germany). Subsequent to homogenization, the tube is spun at 1000 g for 1 hour to pellet tissue debris. Whole protein content is then concentrated using known methods in the art.

In one specific embodiment, concentration of placental proteins is accomplished through solid phase extraction columns such as Sep-Pak C₁₈ cartridges from Waters Corp. In another embodiment, the supernatant is lyophilized and concentrated in a >1000 Da dialysis tube for desalting. The purified proteins are then injected into a patient at a concentration between 1,125 ug to 2,000 ug based on previous work on tumor immunization. See, e.g., Hollinshead, A. C., T. H. Stewart, and R. B. Herberman, Delayed-hypersensitivity reactions to soluble membrane antigens of human malignant lung cells. J Natl Cancer Inst, 1974. 52(2): p. 327-38. Injection schedules include monthly injections in a subcutaneous manner such as in the deltoid region of the arm.

The injection protocol may be adjusted according to the need of the patient. In addition, immunization may be performed through other methods known in the art including via injection (e.g., subcutaneous, intradermal, intramuscular), aerosol, oral, transdermal, intrathecal, transmucosal, intrapleural, or routes commonly used. Modifications to the vaccine preparation can include co-administration with an adjuvant or utilization of the protein preparation as an antigenic source for pulsing DC, and using the pulsed DC as a vaccination preparation.

Another embodiment of the invention is administration of purified porcine trophoblasts as a vaccination source. Said trophoblasts may be purified according to methods known in the art. One method is: chunks of placental cotyledons approximately 30 g are rinsed in saline, separated from membranes and connective tissue, then coarsely minced with scissors and transferred to calcium and magnesium free hanks with 0.185% trypsin, 25 mM HEPES and 0.4 mg/ml type I DNAse. Tissue is then incubated in a water bath at 37 Celsius for 30 minutes with shaking every 5 minutes. Aliquots of the supernatant are then layered over calf serum and centrifuged at 1000 g for 5 minutes at room temperature. Pellets are then layered on Percoll gradient and purified using approximately the 35% Percoll level as reported in Reis, F. M., et al., Corticotropin-releasing factor, urocortin and endothelin-1 stimulate activin A release from cultured human placental cells. Placenta, 2002. 23(6): p. 522-5. Said trophoblasts are approximately 95% pure as determined by staining with cytokeratin, vimentin and CD45. Trophoblasts may be injected into cancer patients at approximately 10⁷-10⁸ cells as needed. Alternatively, purified trophoblasts may be utilized as a source of xenogeneic protein for vaccination. Protein extraction is well described in the art and can be performed by freeze-thaw cycles or through the use of a homogeniser.

Other trophoblastic cell lines have been generated from a variety of species including mouse, pig, and human. Utilization of these alternative cell lines in xenogeneic recipients represents other embodiments of the invention. Cell lines may be irradiated in order to inhibit proliferation and outgrowth. In order to increase immunogenecity, the trophoblast cell lines are induced to express immune stimulatory molecules through pre-treatment with interferon gamma or other agents known to increase expression of immunogenic molecules such as MHC II on cells. Immunogenecity of cell lines can be increased through methods known in the art such as: (1) Transfection with immune-stimulatory cytokines or membrane proteins such as IL-1, IL-2, IL-4, IL-12, TNF-alpha, GM-CSF, or MHC I, MHC II, CD40, CD80, CD86, respectively; (2) Administration of a “stressor” such as hyperthermia or free radical stress; and/or (3) Inhibition of endogenously expressed immune suppressive molecules such as IL-10.

Utilization of mRNA from placental extracts for transfection of DC yields another method of practicing the invention disclosed. RNA from placental tissue can be extracted using methods known in the art such as, utilization of the Trizol reagent. Purified RNA transfected into DC begins to translate antigenic proteins that become processed by the DC through the endogenous pathway. This method has been utilized by Gilboa's group for generation of tumor-vaccines using tumor-derived RNA. Transfected DC can subsequently be induced to mature using methods known in the art such as administration of LPS and/or TNF-alpha, and/or toll-like receptor (TLR) agonists such as Poly (IC). Matured or non-matured DC can subsequently be injected into the cancer patient for stimulation of immunity.

Although the anti-xenogeneic response is highly potent and robust, addition of adjuvants may further increase immunogenecity of the placental preparation. Adjuvants suitable for stimulation of immunity are well-known to the artisan and include co-administration with BCG, De-Tox or unmethylated cpg motifs.

In one embodiment, the invention provides a means of generating a population of cells with tumoricidal ability. Specifically, 50 ml of peripheral blood is extracted from a cancer patient and peripheral blood monoclear cells (PBMC) are isolated using the Ficoll Method. PBMC are subsequently resuspended in 10 ml STEM-34 media and allowed to adhere onto a plastic surface for 2-4 hours. The adherent cells are then cultured at 37° C. in STEM-34 media supplemented with 1,000 U/mL granulocyte-monocyte colony-stimulating factors and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7.

In specific embodiments, the generated DC are used to stimulate T cell and NK cell tumoricidal activity by co-incubation with xenogenic antigens. Sources of xenogenic antigens include, but are not limited to, xenogeneic tumors, or extracts thereof. In various embodiments, trophoblast extracts are used to pulse human dendritic cells in vitro to activate T cells and or NK cells. Trophoblast extracts are generated by lysis of placental tissue, or placental tissue purified to contain high concentrations of trophoblasts. More specifically, trophoblasts are cultured and expanded in vitro before use as antigens.

In addition to human placenta, porcine, murine, canine, bovine, and equine trophoblasts may be used. In one specific embodiment, trophoblasts are isolated from a placenta of a mammal that possesses a hemochorial placenta. Specifically, generated DC may be further purified from culture through use of flow cytometry sorting or magnetic activated cell sorting (MACS), or may be administered to patients as a semi-pure population. DC may be added into said patient in need of therapy with the concept of stimulating NK and T cell activity in vivo, or in another embodiment may be incubated in vitro with a population of cells containing T cells and/or NK cells.

In various specific embodiments, DC are exposed to agents capable of stimulating maturation in vitro. Non-limiting means of stimulating in vitro maturation include culturing DC or DC containing populations with a toll like receptor agonist. Another means of achieving DC maturation involves exposure of DC to TNF-alpha at a concentration of approximately 20 ng/mL.

In order to activate T cells and/or NK cells in vitro, cells are cultured in media containing approximately 1000 IU/ml of interferon gamma. Incubation with interferon gamma may be performed for the period of 2 hours to the period of 7 days. In a preferred embodiment, incubation is performed for approximately 24 hours, after which T cells and/or NK cells are stimulated via the CD3 and CD28 receptors in the presence of placental antigens. The placental antigens are allogeneic, syngeneic, or xenogeneic. One means of accomplishing this is by addition of antibodies capable of activating these receptors.

In one exemplary embodiment, approximately, 2 ug/ml of anti-CD3 antibody is added, together with approximately 1 ug/ml anti-CD28. In order to promote survival of T cells and NK cells and stimulate proliferation, a T cell/NK mitogen is used. In one specific embodiment the cytokine IL-2 is utilized. One example of an IL-2 concentration useful for the practice of the invention is about 500 u/mL. Media containing IL-2 and antibodies are changed every 48 hours for approximately 8-14 days. In one particular embodiment DC are included to the T cells and/or NK cells in order to endow cytotoxic activity towards tumor cells. In a particular embodiment, inhibitors of caspases are added in the culture so as to reduce rate of apoptosis of T cells and/or NK cells. Generated cells are administered to a subject through the appropriate means, including but not limited to intradermally, intramuscularly, subcutaneously, intraperitoneally, intraarterially, intravenously (including indwelling a catheter), intratumorally, or into an afferent lymph vessel.

In one embodiment, the stimulation of immunity to tumors by xenogeneic immunization may be combined with known immune stimulators such as IL-2. When hyperimmunization may lead to adverse events, the utilization of intravenous ascorbic acid (AA) together with immune stimulation is contemplated by the invention provided herein. AA has beneficial effects on the process of systemic inflammation. For example, one mouse study demonstrated that after challenge with the bacteria Klebsiella pneumonia to induce a sepsis-like state, a 3-fold higher mortality was observed in ascorbate-deficient animals compared to controls. See, e.g., Gaut, J. P., et al., Vitamin C fails to protect amino acids and lipids from oxidation during acute inflammation. Free Radic Biol Med, 2006. 40(9): p. 1494-501; and Tyml, K., F. Li, and J. X. Wilson, Septic impairment of capillary blood flow requires nicotinamide adenine dinucleotide phosphate oxidase but not nitric oxide synthase and is rapidly reversed by ascorbate through an endothelial nitric oxide synthase-dependent mechanism. Crit Care Med, 2008. 36(8): p. 2355-62.

Other studies demonstrated that hyper-supplementation with AA resulted in better outcomes in sepsis-associated hypoglycemia, microcirculatory abnormalities, and blunted endothelial responsiveness in animal models. See, e.g., Shen, K. P., et al., Antioxidant eugenosedin-A protects against lipopolysaccharide-induced hypotension, hyperglycaemia and cytokine immunoreactivity in rats and mice. J Pharm Pharmacol, 2005. 57(1): p. 117-25; Tyml, K., F. Li, and J. X. Wilson, Delayed ascorbate bolus protects against maldistribution of microvascular blood flow in septic rat skeletal muscle. Crit Care Med, 2005. 33(8): p. 1823-8; Wu, F., K. Tyml, and J. X. Wilson, Ascorbate inhibits iNOS expression in endotoxin- and IFN gamma-stimulated rat skeletal muscle endothelial cells. FEBS Lett, 2002. 520(1-3): p. 122-6; Wu, F., J. X. Wilson, and K. Tyml, Ascorbate inhibits iNOS expression and preserves vasoconstrictor responsiveness in skeletal muscle of septic mice. Am J Physiol Regul Integr Comp Physiol, 2003. 285(1): p. R50-6; and Wu, F., J. X. Wilson, and K. Tyml, Ascorbate protects against impaired arteriolar constriction in sepsis by inhibiting inducible nitric oxide synthase expression. Free Radic Biol Med, 2004. 37(8): p. 1282-9.

It is within the scope of the current invention to utilize these findings to optimize the therapeutic effects of the immune stimulatory preparations disclosed herein. Further support for the use of AA comes from randomized clinical trials that have been performed in septic patients (sepsis representing a state of immune hyperactivation) using AA and vitamin E, which demonstrated superior outcomes, as well as reduction in parameters of oxidative stress.

One study in the recent history assessed AA alone in patients with systemic inflammation. The investigators examined burn patients with >30% of their total body surface area affected. Patients were administered intravenous AA i.v. (66 mg/kg/hr for 24 hours, n=19) or received only standard care (controls, n=18). AA treatment resulted in statistically significant reductions in 24 hr total fluid infusion volume, and fluid retention (indicative of vascular leakage). Perhaps most striking was the decrease in the need for mechanical ventilation: the treated group required an average of average of 12.1±8.8 days, while the control group required 21.3±15.6 days. See, e.g., Tanaka, H., et al., Reduction of resuscitation fluid volumes in severely burned patients using ascorbic acid administration: a randomized, prospective study. Arch Surg, 2000. 135(3): p. 326-31.

Given that numerous inflammatory markers associated with vascular leak syndrome are also found in SIRS and severe burn patients, the possibility is presented that AA may exert some beneficial effects on IL-2 therapy, both from the reduction of toxicity perspective, as well as from the stimulation of efficacy.

In some embodiments of the invention, the culture of xenoreactive immune cells is performed by starting with purified lymphocyte populations. For example, the step of separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Non-limiting examples of the surface markers include CD3, CD8 and D4, and separation methods depending on these surface markers are known in the art.

For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads, Dynabeads M450 CD8, and Eligix anti-CD8 mAb coated nickel particles are suitably used.

This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activities in terms of tumor killing, as well as migration to the site of the tumor. For example, the step of culturing the cell population and cell sub-population containing a T cell can be performed by selecting suitable known culturing conditions depending on the cell population.

In addition, in the step of stimulating the cell population using xenogeneic extracts from cancer cells, trophoblasts, or trophoblast like cells, are added to the medium to perform culturing. For example, cytokines, chemokines or other ingredients may be added to the medium. Herein, the cytokine is not particularly limited as far as it can act on the T cell, and examples thereof include IL-2, IFN-gamma., transforming growth factor (TGF)-beta, IL-15, IL-7, IFN-alpha., IL-12, CD40L, and IL-27.

From the viewpoint of enhancing cellular immunity, in particular embodiments, IL-2, IFN-gamma, or IL-12 are used and, from the viewpoint of improvement in survival of a transferred T cell in vivo, IL-7, IL-15 or IL-21 are used. In addition, the chemokine is not limited as far as it acts on the T cell and exhibits migration activity. Non-limiting examples thereof include RANTES, CCL21, MIP1alpha, MIP1beta, CCL19, CXCL12, IP-10 and MIG. The stimulation of the cell population can be performed by the presence of a ligand for a molecule present on the surface of the T cell, for example, CD3, CD28, or CD44 and/or an antibody to the molecule.

Further, the cell population can be stimulated by contacting with other lymphocytes such as antigen presenting cells (dendritic cell) presenting a target peptide such as a peptide derived from a cancer antigen on the surface of a cell. In one particular embodiment trophoblasts are used to stimulate T cells and or NK cells to proliferate or be sensitized in vitro before in vivo administration. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide.

Examples of additional methods for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxicity assay, measurement of cytokine mRNA by a quantitative reverse transcriptase polymerase chain reaction, or an assay which is currently used for measuring a T cell response such as a limiting dilution method.

In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide.

Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

One potential dose-limiting toxicities of xenogeneic immunization may be vascular leak syndrome (VLS). This is considered to be the major dose-limiting adverse effect of other immunotherapies such as IL-2 administration. In a meta-analysis of studies performed in metastatic renal cell carcinoma patients, objective responses were observed in 23% of patients, the majority of which lasted more than 10 years. Unfortunately, 65% of patients have to interrupt or stop therapy because of VLS. This syndrome is characterized by an increased extracellular fluid extravasation, hypotension, ascites, pulmonary edema, and hydrothorax, and clinically resembles the systemic inflammatory response syndrome (SIRS). Dermatological manifestations include erythrematous eruptions and mild papillary edema associated with burning and pruritus of the skin.

Severe forms of VLS are associated with pulmonary or cardiac failure with approximately 1% of treated patients having lethal outcome. Typically the symptoms of VLS are treated by vasopressor therapy and judicious fluid replacement, such as with colloid solutions for their osmotic effects. Patients may also be treated with theophylline and terbutaline, for which clinical experience suggests a possible reduction of the severity and frequency of acute episodes.

At a cellular level it is well-known that VLS is associated with endothelial cell activation and increased vascular permeability. Biopsies of patients receiving IL-2 revealed an increased expression of adhesion molecules such as ICAM and LFA-1. These proteins are known to promote granulocyte extravasation, however, such upregulation was not observed when IL-2 was added directly to endothelial cell cultures in vitro, suggesting the effect was mediated by other host components. Given that one of the main cellular targets of IL-2 is the T cells, which express two types of IL-2 receptor, it appears that the initial T cell activation is a major contributor to downstream inflammatory effect on endothelium subsequent to IL-2 administration.

In an early study, Rosenberg's group established a murine model for quantifying VLS by administering radioactively iodinated albumin into mice receiving IL-2 and assessing radioactivity of tissues. In this model, increased gamma-counts are correlated with endothelial permeability and leakage of albumin into tissues. They found that administration of IL-2 to nude mice or mice that have been immune suppressed by radiation, cyclophosphamide, or steroids, was associated with markedly reduced or no vascular leakage. See, e.g., Rosenstein, M., S. E. Ettinghausen, and S. A. Rosenberg, Extravasation of intravascular fluid mediated by the systemic administration of recombinant interleukin 2. J Immunol, 1986. 137(5): p. 1735-421. Accordingly, the process of LAK generation may be involved in stimulation of VLS.

Interestingly, investigators have found that the depletion of host lymphocytes reduced vascular leakage only in response to IL-2 alone, but not in response to IL-2 and LAK transfer. Other studies have shown that cells bearing the NK marker asialo-GM1 are associated with some of the IL-2 associated toxicities. One group showed that antiserum to asialo GM1 suppressed mortality, vascular leak syndrome, and hepatic damage and reduced infiltration of pulmonary and hepatic vasculature by asialo GM1+ lymphocytes induced by IL-2 treatment. Depletion of the Asialo-GM1 bearing cells did not alter lymphoid hyperplasia, tissue infiltration by Lyt 2+ lymphocytes, tissue and peripheral eosinophilia, or thrombocytopenia. Interestingly, the antisera did not affect the anti-tumor efficacy of IL-2 therapy in BDF mice bearing the colon 38 adenocarcinoma. Thus, T cell and NK cell activation by the high dose IL-2 may induce the production of various cytokines, including, but not limited to TNF-alpha, which is known to induce endothelial cell activation locally, and systemically are mediators of SIRS. The knowledge of inflammatory cascades induced by hyperactivation of the immune system in cancer is useful for one of skill in the art to practice the invention in a manner in which adverse effects are minimized while therapeutic effects are maintained.

Another event associated with administration of potent immune stimulators such as IL-2 is complement activation. The complement system is an enzymatic cascade of about 30 circulating proteins, primarily generated by the liver that cause inflammation and amplification of a various immune responses. The complement system can be activated through the classical (antibody mediated) pathway, alternative pathways (antibody-independent), or through the mannose-binding lectin pathway, all leading to formation of the membrane attack complex which causes cellular lysis through generation of pores in the membrane.

In a clinical study of metastatic renal cancer patients receiving IL-2 via a 24-hour i.v. infusion at a daily dose of 3×10(6) U/m2 for 5 consecutive days, the classical complement pathway components C3 and C4 were measured daily during IL-2 infusion, and after its interruption. IL-2 administration was associated with a significant decrease in both C3 and C4 levels, which normalized on average 5 days after the end of IL-2 infusion. See, e.g., Lissoni, P., et al., Activation of the complement system during immunotherapy of cancer with interleukin-2: a possible explanation of the capillary leak syndrome. Int J Biol Markers, 1990. 5(4): p. 195-7.

Another study associated presence of VLS in patients receiving IL-2 with complement activation as assessed by levels of C3a and the classical complement component C4a. In this study, levels of C3a were as elevated as those found in septic and burn patients. See, e.g., Thijs, L. G., et al., Activation of the complement system during immunotherapy with recombinant IL-2. Relation to the development of side effects. J Immunol, 1990. 144(6): p. 2419-24.

Another study examining 23 cancer patients undergoing therapy with interleukin-2 and lymphokine-activated killer cells demonstrated 3-fold elevations of C3a desArg concentrations by the 8th day of therapy with concentrations of C4a desArg also being elevated by the end of therapy. Associated with activation of the complement system was an increase in the neutrophil cell-surface expression of complement receptor Type 1 and complement receptor Type 3. See, e.g., Moore, F. D., Jr., et al., The systemic complement activation caused by interleukin-2/lymphokine-activated killer-cell therapy of cancer causes minimal systemic neutrophil activation. Int J Cancer, 1991. 49(4): p. 504-8.

An interesting dependence on T cells for complement activation may bridge the studies demonstrating that T cells are necessary for endothelial activation and VLS associated with IL-2 administration. One study showed that cancer patients had pretreatment similar to control plasma levels of C3a, Ba, Bb, and SCSb-9. Post-IL-2 treatment C3a levels where shown to be increased on average of 15.6-fold as compared to controls. The Ba and Bb proteins, which belong to the alternatively complement activation pathway were augmented 8.0-fold and 5.0-fold, respectively, subsequent to IL-2 treatment. The plasma levels of the effector complement complex, SC5b-9, was increased 5.0-fold and the plasma C4d and iC3b concentrations increased 4.8- and 2.9-fold, respectively, after treatment.

To show the involvement of patient lymphocytes in complement activation, the investigators found that cells expressing the T cell marker CD3 had increased surface expression of anti-C3c and anti-SC5b-9 by 6.2-fold and 5.1-fold, respectively after IL-2 therapy. These results lead the investigators to conclude that the T cells were participating in the IL-2 induced complement activation. This was also demonstrated in that increased concentration of the inflammatory protein C-reactive protein (CRP) was found post-IL-2 therapy, and that the T cells bound CRP. T cell bound CRP was capable of activating the alternative complement pathway. See, Deehan, D. J., et al., Correlation of serum cytokine and acute phase reactant levels with alterations in weight and serum albumin in patients receiving immunotherapy with recombinant IL-2. Clin Exp Immunol, 1994. 95(3): p. 366-72; Vachino, G., et al., Complement activation in cancer patients undergoing immunotherapy with interleukin-2 (IL-2): binding of complement and C-reactive protein by IL-2-activated lymphocytes. Blood, 1991. 78(10): p. 2505-13.

Therapeutically, administration of the complement inhibitor C1 esterase inhibitor is capable of reducing IL-2 induced hypotension and complement activation in patients. Various components of the complement cascade directly activate endothelial cells, with endothelial cell activation not only causing lymphocyte and neutrophil extravasation, but also thrombosis by the upregulation of tissue factor. C5a is a by-product of complement activation that has been demonstrated to induce endothelial cell activation and permeability. This protein is also a major effector in systemic inflammatory disorders and antibodies to it are being assessed clinically for this condition with some efficacy signals and suppression of endothelial activation.

The complement effector complex SC5b-9 was demonstrated in vitro to induce endothelial cell activation by stimulating expression of the Response Gene to Complement (RGC)-32, which in turn activates CDC2 and the AKT pathway. Jeffrey Platt's group concluded that complement activation is associated with induction of IL-1, which in turn stimulates endothelial cells expression of E-selectin, intracellular adhesion molecule-1, vascular cell adhesion molecule-1, Ikappa-Balpha, interleukin (IL)-1alpha, IL-1beta, IL-8, and tissue factor. See, Saadi, S., et al., Endothelial cell activation by pore forming structures: pivotal role for interleukin-1alpha. Circulation, 2000. 101(15): p. 1867-73. Thus, in the cascade of IL-2 induced VLS, T cell activation may be associated with complement activation and complement activation in turn stimulates endothelial cell activation.

One of the basic features of endothelial cell activation is stimulation of the clotting cascade. Thus in various specific embodiments of the invention, the protection of endothelial cells from immune associated activation is envisioned. Means of protecting endothelium include administration of intravenous antioxidants such as intravenous ascorbic acid. In general, ascorbic acid or its salts are used in an amount of 5.0 to 30.0 g, preferably in an amount of 5.0 to 10.0 g. The parenteral preparation has a pH between 6.0 and 8.0. Guidance to the use of intravenous ascorbic acid for tumor patients may be found in U.S. Pat. No. 6,284,786 and U.S. Pat. No. 6,426,076.

According to the emerging picture that VLS has many common elements with SIRS, one of the common features is development of local thrombocyte and coagulation system activation. Innate immune response possesses the ability to locally marginalize pathogens by stimulation of clotting and consequent sequestration. However, this process becomes pathological when it occurs at a systemic level, such as SIRS or VLS. Upregulation of tissue factor expression has been seen on endothelial cells from animals treated with IL-2. See, e.g., Trichonas, G., et al., A novel nonradioactive method to evaluate vascular barrier breakdown and leakage. Invest Ophthalmol Vis Sci. 51(3): p. 1677-82.

Expression of this protein is known to cause activation of the clotting cascade, as well as stimulate inflammatory processes. Hack et al demonstrated activation of the contact system of coagulation proteins by showing that patients on IL-2 therapy had degradation of factor XII and prekallikrein. Reductions in these proteins appeared not due to protein leakage into the interstitial space, since their levels were still significantly lower, i.e., 80 and 50%, respectively, when corrected for albumin decreases (endothelium may still be more permeable to these clotting factors than to albumin, that's why i would prefer “appeared”). See, Hack, C. E., et al., Studies on the contact system of coagulation during therapy with high doses of recombinant IL-2: implications for septic shock. Thromb Haemost, 1991. 65(5): p. 497-503.

Accordingly, non-specific activation of the coagulation system and a resulting potential for thrombosis, occurs as a result of IL-2 treatment. Given the inherently pro-thrombotic state of many cancer patients, it is theoretically possible that IL-2 therapy may have thrombotic complications. In certain embodiments of the invention, manipulation of the clotting cascade is performed in conjunction with immunotherapy so as to reduce clotting abnormalities that may arise during immune activation.

Granulocyte activation and tissue infiltrations are hallmarks of systemic immune/inflammatory activation. In a study of 4 patients on IL-2, granulocytes became activated following IL-2 with mean peak elastase/alpha 1-antitrypsin (E alpha 1 A) and lactoferrin values of 212 (SEM=37) and 534 (SEM=92) ng ml-1 respectively occurring 6 h after the IL-2. Activation of the complement cascade was evidenced by a dose dependent elevation of peak C3a values on day 5 of IL-2. There was a significant correlation between C3a levels and the degree of hypotension during the first 24 h after IL-2 (r=0.91) and parameters of capillary leakage such as weight gain and fall in serum albumin (r=0.71), which led to the conclusion that activation of PMN initiates endothelial cell damage subsequently leads to activation of the complement cascade.

Neutrophils of patients on IL-2 therapy expressed both phenotypic (up-regulation of CD11 b/CD18 adhesion receptor expression) and functional (hydrogen peroxide and hypochlorous acid production) evidence of potent neutrophil activation. Accordingly, knowledge of neutrophil activation during induction of hyper-immunity to tumors should be taken into consideration and appropriately dealt with by the use of anti-inflammatories as needed. Associated with chronic inflammatory states such as heart failure, mucositis, and acute states such as sepsis or GVHD, is translocation of bacterial flora into systemic circulation.

Interestingly, an interference with the gut flora and inflammation also appears to be present in IL-2 toxicity. In a recent study, 51 male rats were randomised to receive rIL-2 by intraperitoneal injection at doses (IU) of 10(5) (n=15), 10(4) (n=8), 10(3) (n=8) or 10(2) (n=8) twice daily, or a saline bolus (n=12). After 5 days, ileal histomorphology was assessed and the mesenteric lymph node complex cultured. Results showed that colonisation of mesenteric lymph nodes with Escherichia coli occurred in all rats treated with 10(5) IU of rIL-2, and in 62%, 37% and 12% of rats treated with decreasing doses of rIL-2. No translocation was observed in control animals. An increase in submucosal lymphatics and occasional mucosal disruption was seen only in the group receiving 10(5) IU. This data shows that rIL-2 promotes bacterial translocation and suggests a mechanism that may fuel high-dose rIL-2 toxicity in man. See, Reynolds, J. V., et al., High-dose interleukin 2 promotes bacterial translocation from the gut. Br J Cancer, 1995, 72(3): p. 634-6.

Given the potent effects seen clinically with homeostatically-induced lymphocyte activation, and the recent findings that T cell homeostatic proliferation appears to be associated with gut flora translocation, it is believed that tumor suppressive activity of IL-2 may be highly dependent on the gut flora, thus possibly explaining inter-patient variation. Thus in one embodiment of the invention, manipulation of the gut microbiome by antibiotics or probiotics is contemplated within the context of the current invention. Numerous probiotics have been shown to decrease inflammation including lactic acid bacteria as described in U.S. Pat. Nos. 8,197,805; 5,728,380; 5,589,168 and 7,195,906, each of which are incorporated by reference herein.

Several studies have demonstrated that oxidative stress modifies endothelial cells in a manner to preferentially activate the complement cascade. The involvement of the mannose-binding lectin and the lectin complement pathway (LCP) in promoting complement activation by endothelial cells post oxidative stress was shown in studies using hypoxic (24 hours; 1% O(2))/reoxygenated (3 hours; 21% O₂) human endothelial cells. Using iC3b deposition as a marker of complement activation, it was shown that N-acetyl-D-glucosamine or D-mannose, but not L-mannose, blocked activation, suggesting that oxidative stress upregulates the mannose dependent pathway. This was also demonstrated using mannose binding lectin deficient serum, as well as antibodies to mannose binding lectin. Furthermore C3 deposition was found in ischemic areas in rats that underwent cardiac ischemia reperfusion injury, a known inducer of oxidative stress.

In some embodiments of the invention, administration of a cytotoxic, or other cancer therapeutic is utilized prior to immunization in order to reduce tumor mass. Cancer therapeutics useful in various embodiments of the invention described herein, include, but are not limited to: Aceglatone; Aclarubicin; Altretamine; Aminoglutethimide; 5-Aminogleavulinic Acid; Amsacrine; Anastrozole; Ancitabine Hydrochloride; 17-1A Antibody; Antilymphocyte Immunoglobulins; Antineoplaston A10; Asparaginase; Pegaspargase; Azacitidine; Azathioprine; Batimastat; Benzoporphyrin Derivative; Bicalutamide; Bisantrene Hydrochloride; Bleomycin Sulphate; Brequinar Sodium; Broxuridine; Busulphan; Campath-IH; Caracemide; Carbetimer; Carboplatin; Carboquone; Carmofur; Carmustine; Chlorambucil; Chlorozotocin; Chromomycin; Cisplatin; Cladribine; Corynebacterium parvum; Cyclophosphamide; Cyclosporin; Cytarabine; Dacarbazine; Dactinomycin; Daunorubicin Hydrochloride; Decitabine; Diaziquone; Dichlorodiethylsulphide; Didemnin B.; Docetaxel; Doxifluridine; Doxorubicin Hychloride; Droloxifene; Echinomycin; Edatrexate; Elliptinium; Elmustine; Enloplatin; Enocitabine; Epirubicin Hydrochloride; Estramustine Sodium Phosphate; Etanidazole; Ethoglucid; Etoposide; Fadrozole Hydrochloride; Fazarabine; Fenretinide; Floxuridine; Fludarabine Phosphate; Fluorouracil; Flutamide; Formestane; Fotemustine; Gallium Nitrate; Gencitabine; Gusperimus; Homoharringtonine; Hydroxyurea; Idarubicin Hydrochloride; Ifosfamide; Ilmofosine; Improsulfan Tosylate; Inolimomab; Interleukin-2; Irinotecan; JM-216; Letrozole; Lithium Gamolenate; Lobaplatin; Lomustine; Lonidamine; Mafosfamide; Meiphalan; Menogaril; Mercaptopurine; Methotrexate; Methotrexate Sodium; Miboplatin; Miltefosine; Misonidazole; Mitobronitol; Mitoguazone Dihydrochioride; Mitolactol; Mitomycin; Mitotane; Mitozanetrone Hydrochloride; Mizoribine; Mopidamol; Muitlaichilpeptide; Muromonab-CD3; Mustine Hydrochloride; Mycophenolic Acid; Mycophenolate Mofetil; Nedaplatin; Nilutamide; Nimustine Hydrochloride; Oxaliplatin; Paclitaxel; PCNU; Penostatin; Peplomycin Sulphate; Pipobroman; Pirarubicin; Piritrexim Isethionate; Piroxantrone Hydrochloride; Plicamycin; porfimer Sodium; Prednimustine; Procarbazine Hydrochloride; Raltitrexed; Ranimustine; Razoxane; Rogletimide; Roquinimex; Sebriplatin; Semustine; Sirolimus; Sizofiran; Sobuzoxane; Sodium Bromebrate; Sparfosic Acid; Sparfosate Sodium; Sreptozocin; Sulofenur; Tacrolimus; Tamoxifen; Tegafur; Teloxantrone Hydrochloride; Temozolomide; Teniposide; Testolactone; Tetrasodium Mesotetraphenylporphine-sulphonate; Thioguanine; Thioinosine; Thiotepa; Topotecan; Toremifene; Treosulfan; Trimetrexate; Trofosfamide; Tumor Necrosis Factor; Ubenimex; Uramustine; Vinblastine Sulphate; Vincristine Sulphate; Vindesine Sulphate; Vinorelbine Tartrate; Vorozole; Zinostatin; Zolimomab Aritox; and Zorubicin Hydrochloride.

The following examples are provided to further illustrate the embodiments of the present invention, but are not intended to limit the scope of the invention. While they are typical of those that might be used, other procedures, methodologies, or techniques known to those skilled in the art may alternatively be used. It is to be understood that modifications of the disclosed invention that do not significantly or substantially affect the result of the invention are covered within the means of the present disclosure. The examples provided below are not meant to restrict the scope of the invention but to illustrate specific embodiments.

### Example 1

### Prophylactic Administration of Xenogeneic Placenta Vaccine

### Porcine Tissue and B16 Lysate Preparation:

Porcine placental tissue was obtained from delivering sows and washed in sterile phosphate buffered saline (PBS) containing 5% penicillin streptomycin mixture and placed on ice for transportation. Placental tissue was homogenized with a tissue grinder and exposed to 4 freeze-thaw cycles alternating from liquid nitrogen to 42 Celsius water bath. Cell debris was pelletted by centrifugation at 1500 g for 45 minutes. Supernatant was collected and sterilized with 0.2 micron Millipore filters. Total protein concentration was determined using the Bradford Assay (BioRad). For control tissue, porcine liver and B16 melanoma cell line proteins were isolated using identical protocol.

### Vaccine Preparation and Administration:

The whole protein preparations were dissolved into sterile, injection-grade PBS at a concentration of 2 mg/ml, and injections of 50 uL (total mass 10 ug) were performed subcutaneously into C57/BL6 mice 7 days before tumor challenge.

### Tumor Model

5×10⁵ B16 American Type Culture Collection (Manassas, Va.) cells were injected subcutaneously into the hind limb flank of female 6-8 week-old C57BL/6 (The Jackson Laboratory: Bar Harbor, Me.). Tumor growth was assessed every 3 days by two measurements of perpendicular diameters by a caliper, and animals were sacrificed when tumors reached a size of 1 cm in any direction. Tumor volume was calculated by the following formula: (the shortest diameter²×the longest diameter)/2

### Experimental Groups

Four (4) C57/BL6 mice per group received the following treatment 7 days before tumor challenge:
A. 50 μl saline treatment
B. 10 μg porcine liver extracts in 50 μL saline
C. 10 μg porcine placenta extracts in 50 μL saline
D. 10 μg B16 melanoma cell extracts in 50 μL saline

### Results:

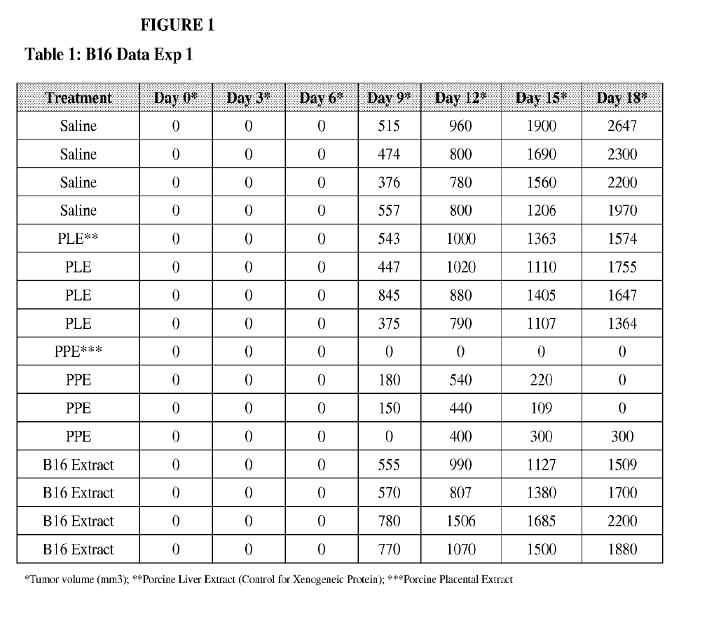

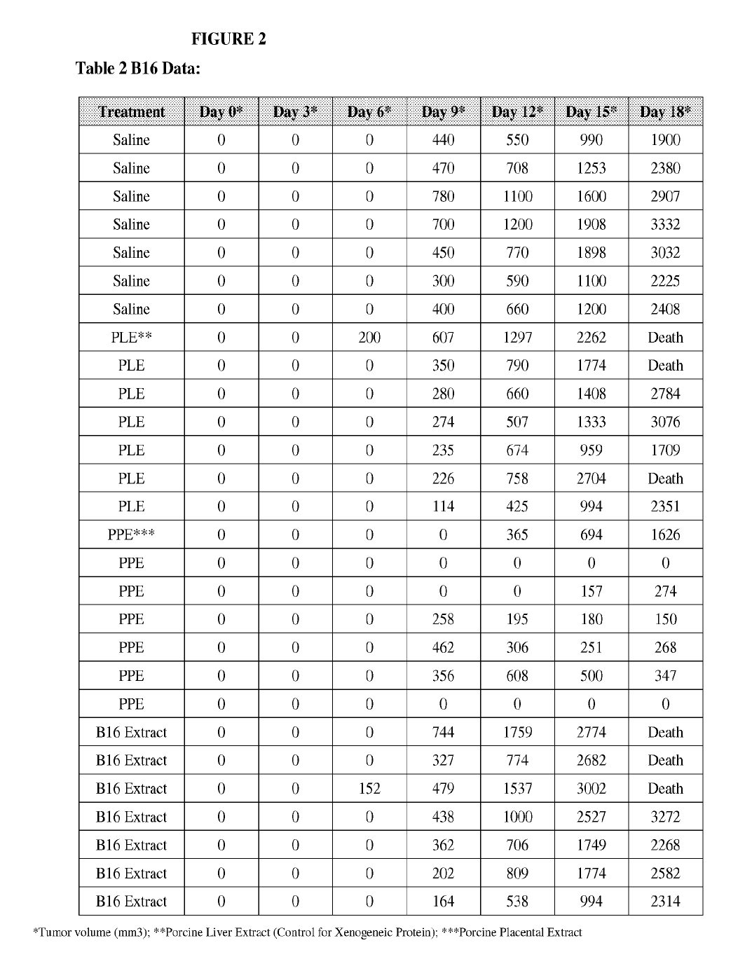

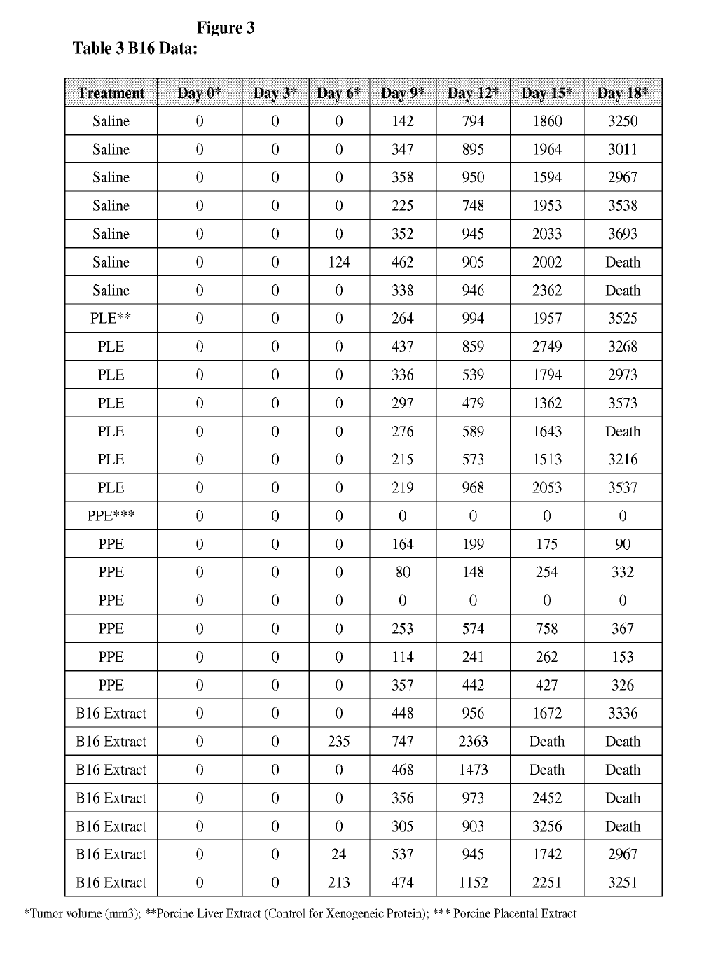

As illustrated in FIGS. 1-3, a statistically-significant inhibition of B16 melanoma cell growth was noted in mice receiving porcine placental extract but not the control porcine liver extract. This indicates the tissue-specific cancer immunization abilities of xenogeneic placenta.

### Example 2

### Xenogeneic Placenta Vaccination Using Autologous Dendritic Cells as Adjuvants

To further illustrate the invention, placental protein extracts were purified as described in the above example. Using B16 melanoma-bearing C57/BL6 mice as tumor models a source of syngeneic DC was needed. Briefly, bone marrow cells were flushed from the femurs and tibias of C57/BL6 mice (Jackson Labs, Bar Harbor Me.), washed and cultured in 24-well plates (2×10⁶ cells per well) in 2 ml of complete medium (RPMI-1640 supplemented with 2 mM L-glutamine, 100 U/ml of penicillin, 100 rig of streptomycin, 50 μM 2-mercaptoethanol, and 10% fetal calf serum (all from Gibco RBL)) supplemented with recombinant GM-CSF (10 ng/ml; Peprotech, Rocky Hill, N.J.) and recombinant mouse IL-4 (10 ng/ml; Peprotech). All cultures were incubated at 37° C. in 5% humidified CO₂. Non-adherent granulocytes were removed after 48 hrs of culture and a fresh medium was added.

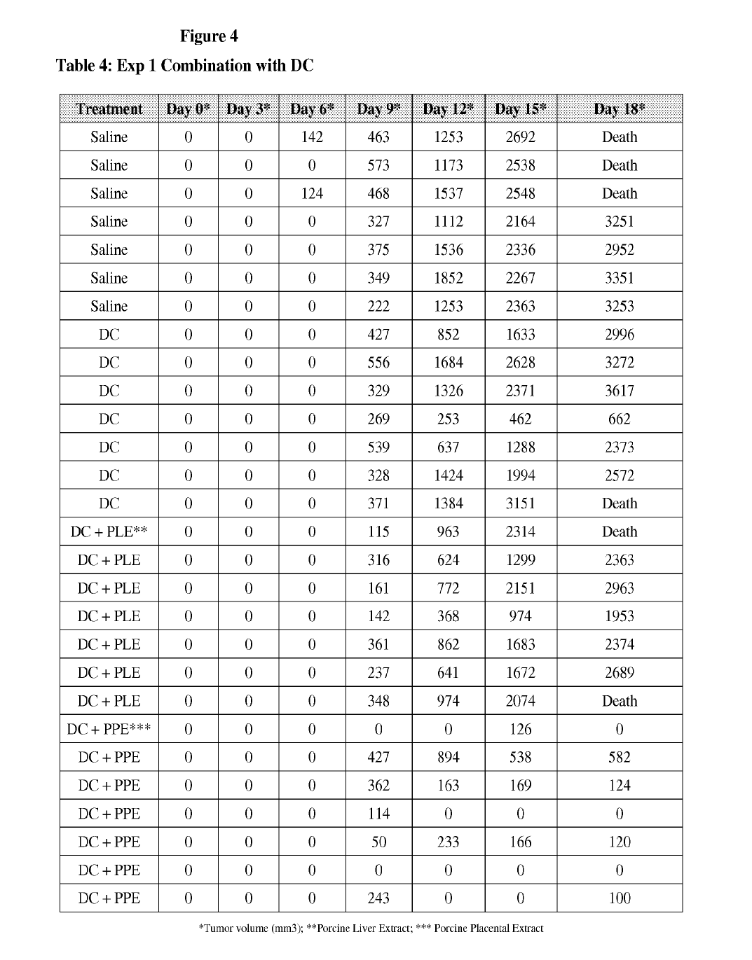

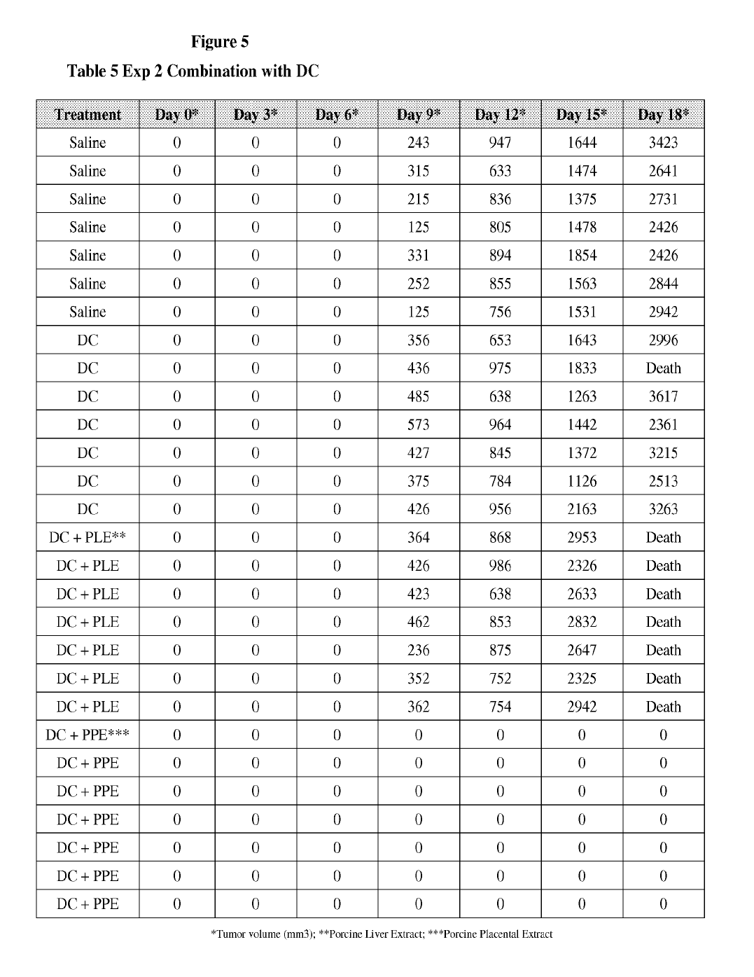

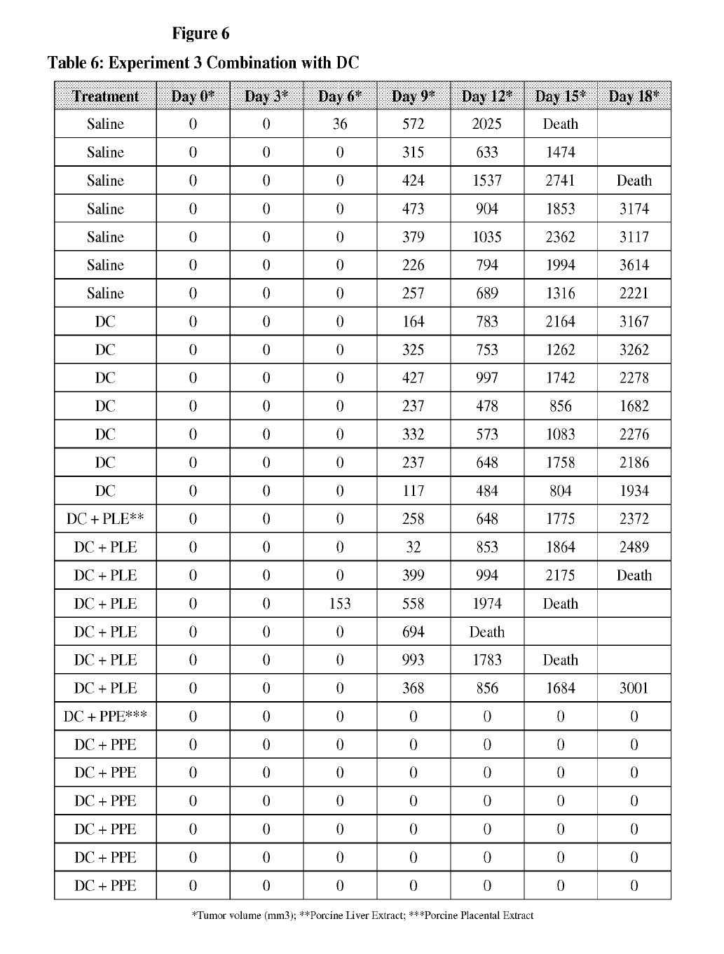

After 7 days of culture >90% of the cells expressed characteristic DC specific markers as determined by FACS. DC were washed and plated in 24-well plates at a concentration of 2×10⁵ cells per well in 400 μl of serum-free RPMI-1640. Day 7 bone marrow-derived DC were either left unpulsed, or pulsed with 10 μg/ml porcine liver homogenate (extract) or xenogeneic porcine placental homogenate (extract). For pulsing, DC was incubated for 24 hours with liver or placental extracts. Subsequently DC was administered at a concentration of 500,000 cells per mouse subcutaneously. A concurrent injection of 5×10⁵ B16 melanoma cells was administered as described above. For controls, porcine liver homogenate and DC alone were administered. As illustrated in FIGS. 4, 5 and 6, none of the mice receiving the DC pulsed with xenogeneic placenta developed significant tumors.

### Example 3

### Xenogeneic Placenta Vaccination can Increase Immunogenecity of Tumor Cell Lysate Vaccine

A potential concern for the utilization of xenoplacental vaccination is the lack of tumor-tissue specificity. In light of the immunostimulatory properties of placental vaccination, we questioned whether co-administration of the xenogeneic vaccine preparation can augment immunogencity of an existing cancer vaccination source.

C57/B6 mice were administered B16 melanoma as described in Example 1. Subsequently mice were treated on the same day with: (a) Saline; (b) B16 lysate; (c) B16 lysate together with porcine liver lysate; (d) B16 lysate together with porcine placental lysate; (e) porcine liver lysate alone; and (f) porcine placental lysate alone

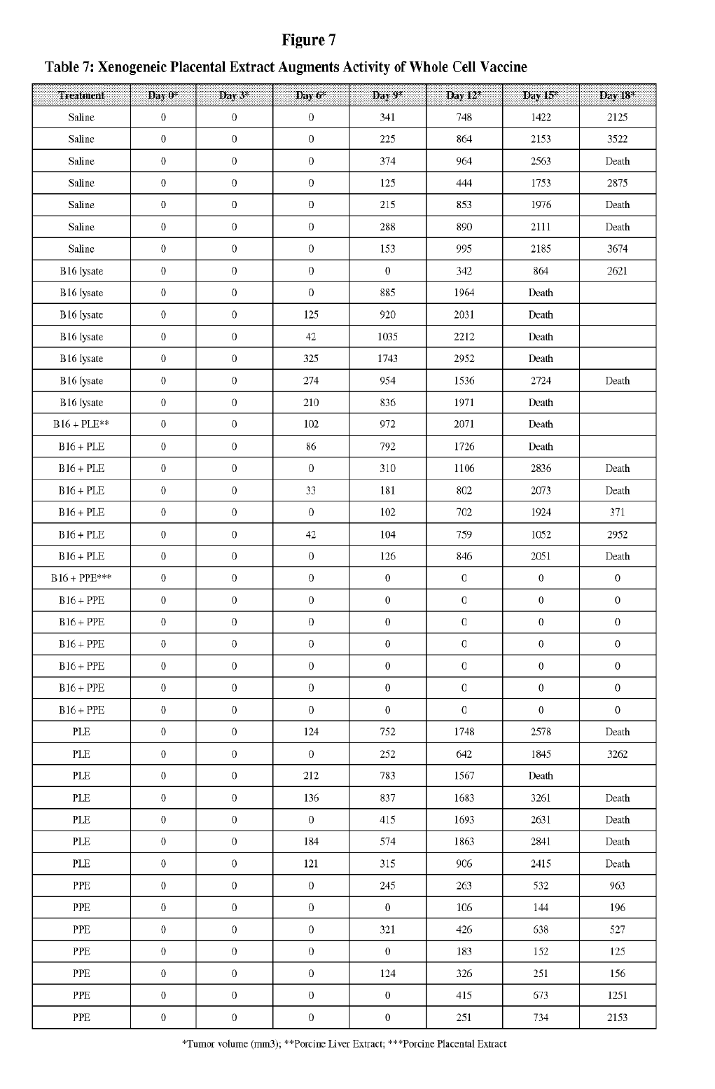

When mice were inoculated with tumor cells at the same time as the vaccine preparation, there was no protection when the melanoma B16 cells were immunized alone. In contrast immunization with the co-mix had a more potent effect than the xenogeneic placenta alone. Notably, the placenta alone had some tumor-protective activities (FIG. 7).

### Example 4

### Treatment of Colon Cancer by Xenoplacental Vaccination

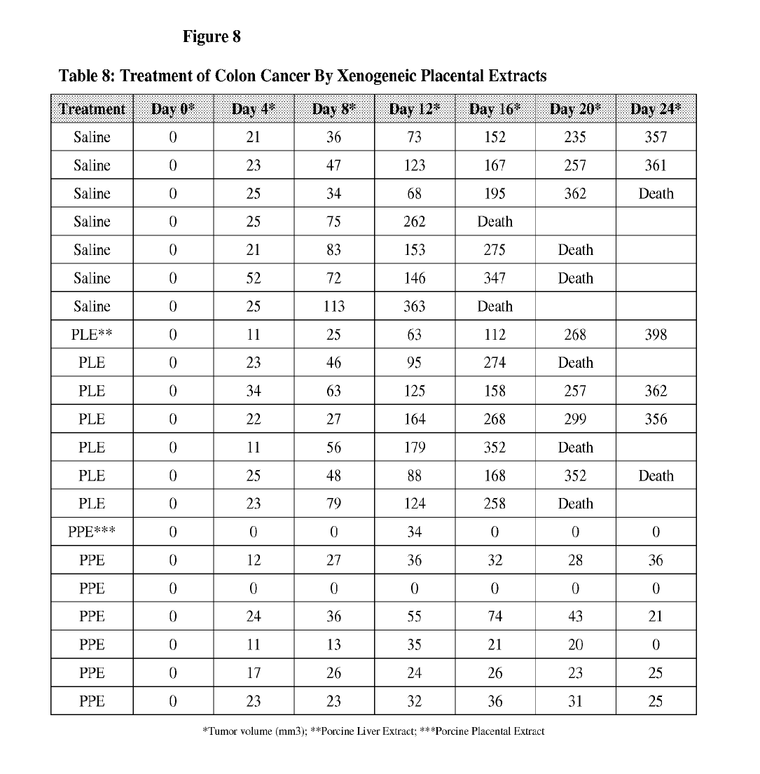

6-8 week old female C57/B6 mice were injected with 200,000 MC38 colon cancer cells subcutaneously in the flank area. Animals were treated with either: (a) saline; (b) porcine liver extracts (PLE); and (c) porcine placenta extract (PPE) on day 5, 10, and 15 after tumor challenge. (2×10(5)) MC38 murine colon adenocarcinoma Cells, tumors measured 2 times per week. As shown in FIG. 8 xenogeneic placental extracts (porcine) blocked tumor growth.

While certain embodiments have been described above, it will be understood that the embodiments described are by way of example only. Accordingly, the systems and methods described herein should not be limited based on the described embodiments. Rather, the compositions and methods described herein should only be limited in light of the claims that follow when taken in conjunction with the above description and accompanying drawings.

All references cited herein, including patents, patent applications, and publications, are hereby incorporated by reference in their entireties, whether previously specifically incorporated or not.

While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

1. A composition comprised of placenta or placental extracts suitable for immunization of a xenogeneic host for treatment of an immune disorder associated with suppression of immune response.

2. The composition of claim 1, wherein the placenta is substantially homogenized and isolated cells are used for vaccination.

3. The composition of claim 2, wherein the isolated cells are trophoblasts, trophoblastic, syncitial trophoblasts, and endothelial cells

4. The composition of claim 1, further comprising an antigen presenting cell.

5. The composition of claim 4, wherein the antigen presenting cell is a dendritic cell.

6. The composition of claim 1, wherein the immune disorder is cancer, a human immunodeficiency virus, chronic fatigue syndrome, or a virally-induced immune suppression.

7. A method for the treatment of cancer comprising administration of xenogeneic placenta into a recipient in need thereof.

8. The method of claim 7, wherein the placenta is substantially homogenized and isolated cells or products thereof are used for vaccination.

9. The method of claim 8, wherein the isolated cells are trophoblasts, trophoblastic, syncitia trophoblasts or endothelial cells.

10. The method of claim 7, further comprising administration of an antigen presenting cell.

11. The method of claim 10, wherein the antigen presenting cell is a dendritic cell.

12. The method of claim 7, wherein the origin of the placental preparation is porcine and the recipient is a human.

13. The method of claim 7, wherein the placental vaccination is administered with an adjuvant to increase immunogenecity.

14. (canceled)

15. A method of treating cancer comprising of immunization of the patient in need with a composition comprising: xenogeneic placenta protein extracts, xenogeneic placentally-derived cells, xenogeneic trophoblasts, or xenogeneic trophoblasts fused with allogeneic or xenogeneic tumor cells.

16. The method of claim 15, wherein an adjuvant is added.

17. The method of claim 16, wherein the adjuvant is a chemical, composition of chemicals, a bacterially-derived compound, or an agonist of immune stimulatory receptors.

18. The method of claim 17, wherein the adjuvant is selected from a group consisting of DETOX, IL-12, CpG-oligodeoxynucleotides, CTLA-4 blockade, JPN-alpha 2b, CCR5 blockade, aluminum hydroxide, montanide ISA 51, GM-CSP,

19. The method of claim 17, wherein the adjuvant is a dendritic cell.

20. The method of claim 19, wherein the adjuvant is a dendritic cell transfected with genes selected from a group consisting of 4-1BBL, IL-2, IL-12, GM-CSP, OX-40 ligand, JPN-gamma, heat shock protein 70, and chaperone-rich cell lysate.
